SEQUENCE LISTING (4 sequences)

SEQ ID NO: 1 - protein, 374 aa, synthetic construct
  REGION 1..374: note = full-length CD64
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 2 - protein, 632 aa, synthetic construct
  REGION 1..632: note = fCD64_mGFP vector
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGATGGGGSGGGGSGGGGSGGGGSMSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITHGMDELYK

SEQ ID NO: 3 - protein, 607 aa, synthetic construct
  REGION 1..607: note = IgK_ecto_TM_mGFP vector
METDTLLLWVLLLWVPGSTGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHGGGGSGGGGSGGGGSGGGGSIIPIVAGVVAGIVLIGLALLLIWKLLMIIHDRGGGGSGGGGSGGGGSGGGGSMSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITHGMDELYK

SEQ ID NO: 4 - protein, 607 aa, synthetic construct
  REGION 1..607: note = IT_ecto_TM_mGFP vector
MNLQPIFWIGLISSVCCVFAQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHGGGGSGGGGSGGGGSGGGGSIIPIVAGVVAGIVLIGLALLLIWKLLMIIHDRGGGGSGGGGSGGGGSGGGGSMSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITHGMDELYK

(19) United States
(12) Patent Application Publication
(10) Pub. No.: US 2024/0018211 A1
(43) Pub. Date: Jan. 18, 2024

(54) EXOSOME COMPRISING OVEREXPRESSED FC RECEPTOR OR PORTION THEREOF, AND METHOD FOR PREPARING SAME

(71) Applicant: EXOCOBIO INC, Cheongju-si (KR)

(72) Inventor: Byong Seung CHO, Gunpo-si (KR)

(73) Assignee: EXOCOBIO INC, Cheongju-si (KR)

(21) Appl. No.: 18/329,960

(22) Filed: Jun. 6, 2023

Related U.S. Application Data
(63) Continuation of application No. PCT/KR2021/019898, filed on Dec. 25, 2021.

(30) Foreign Application Priority Data
Jan. 11, 2021 (KR) 10-2021-0003600
Dec. 16, 2021 (KR) 10-2021-0181002

(51) Int. Cl. C07K 14/735; C12N 5/071; A61P 35/00; C07K 16/28

(52) U.S. Cl. CPC C07K 14/70535; C12N 5/0686; A61P 35/00; C07K 16/2863

(57) ABSTRACT

A method is provided for producing exosomes containing an overexpressed CD64 or a portion thereof. The method includes transducing a genetic construct encoding a full-length CD64 or a portion thereof into cells that do not naturally express CD64 or a portion thereof, without fusion with a nucleic acid sequence encoding an exogenous transmembrane protein or a transmembrane domain thereof; expressing the genetic construct in the cells; culturing the cells in a medium; and isolating exosomes secreted or released into the cell culture medium.

### CROSS REFERENCE

This application is a Bypass Continuation of International Application No. PCT/KR2021/019898 filed Dec. 25, 2021, claiming priority based on Korean Patent Application No. 10-2021-0003600 filed Jan. 11, 2021 and Korean Patent Application No. 10-2021-0181002 filed Dec. 16, 2021, the entire contents of which are incorporated herein by reference.

### INCORPORATION BY REFERENCE OF SEQUENCE LISTING

The content of the electronically submitted sequence listing, file name: Q287749_SEQ_LIS_AS_FILED.xml; size: 8,257 bytes; and date of creation: Jun. 6, 2023, filed herewith, is incorporated herein by reference in its entirety.

### TECHNICAL FIELD

The present invention relates to exosomes containing an overexpressed Fc receptor or a portion thereof and a method for producing the same.

In addition, the present invention relates to exosomes containing an overexpressed Fc receptor or a portion thereof and a method for producing the same, wherein the exosomes are produced by transducing a genetic construct encoding a full-length Fc receptor or a portion thereof into cells that do not naturally express the Fc receptor or the portion thereof, without fusion with a nucleic acid sequence encoding an exogenous transmembrane protein or a transmembrane domain thereof.

Moreover, the present invention relates to the application of exosomes containing an overexpressed Fc receptor or a portion thereof to the prevention, suppression, alleviation, amelioration or treatment of immune-related diseases, cancer, inflammatory diseases, viral diseases, and various other diseases.

### BACKGROUND ART

A drug delivery system (DDS) is used to make a therapeutic agent, administered to a human body, work effectively and to reduce side effects.

For example, when an antibody, a protein, or a polypeptide is exposed to a human environment, it may have reduced efficacy or may not be delivered to a target site to be treated. For example, targeted anticancer drugs have been developed to act specifically on cancer cells. If they could act more selectively on cancer tissues to be treated when administered into a human body, the therapeutic effects thereof could be maximized.

Liposomes or micelles developed so far as drug delivery systems prolong the retention time of drugs in a human body and improve pharmacokinetics, but lack the ability to selectively target specific cells, such as immune cells or cancer cells. In addition, liposomes made from synthetic materials may raise biocompatibility problems.

Recently, there have been reports that cell secretomes contain various bioactive molecules that regulate cellular behaviors. In particular, cell secretomes contain ‘exosome’ or ‘extracellular vesicle’ that has intercellular signaling functions, and thus studies on the components and functions thereof have been actively conducted.

Cells release various membranous vesicles to their extracellular environment, and these released vesicles are usually called extracellular vesicles (EVs). The EV is also called cell membrane-derived vesicle, ectosome, shedding vesicle, microparticle, exosome, etc., and is also used discriminately from exosome in some cases.

Exosome is a vesicle of tens to hundreds of nanometers in size, which comprises a phospholipid bilayer membrane having the same structure as that of the cell membrane. This exosome contains proteins, nucleic acids (mRNA, miRNA, etc.) and the like which are called exosome cargo. It is known that exosome cargo includes a wide range of signaling factors, and these signaling factors are specific for cell types and regulated differently depending on secretory cells' environment. It is known that exosome is an intercellular signaling mediator secreted by cells, and various cellular signals transmitted through it regulate cellular behaviors, including the activation, growth, migration, differentiation, dedifferentiation, apoptosis, and necrosis of target cells. Exosome contains specific genetic materials and bioactive factors depending on the nature and state of cells from which the exosome was derived. Exosome derived from proliferating stem cells regulates cell behaviors such as cell migration, proliferation and differentiation, and recapitulates the function of stem cells involved in tissue regeneration (Nature Review Immunology 2002 (2) 569-579).

That is, exosomes called “avatars” of cells contain bioactive factors such as growth factors, similar to cells, and serve as carriers that transmit bioactive factors between cells, that is, serve to mediate cell-to-cell communication. Exosomes are known to be released not only from animal cells such as stem cells, immune cells, fibroblasts and cancer cells, but also from cells of various organisms such as plants, bacteria, fungi, and algae.

When such exosomes are used as a drug delivery system, there are advantages that they are biocompatible and well absorbed with increasing drug stability in a human body. However, a loading method of passively absorbing drug cargoes into natural exosomes using hydrophobic properties has a limitation that production efficiency is low and sufficient amounts of drug cargoes cannot be loaded into exosomes.

Recently, there has been proposed a method of obtaining target protein or peptide-loaded exosomes (exosomes in which a target protein or peptide is fused to an exosomal transmembrane protein) by fusing a nucleic acid sequence encoding a target protein or peptide to a nucleic acid sequence encoding an exosomal transmembrane protein in a genetic construction preparation step, expressing the prepared genetic construction in cells, and isolating exosomes of interest secreted or released into a cell culture medium (see WO 2013/084000 A2; and WO 2014/168548 A2).

However, like other technical fields, the technical field to which the present invention belongs also requires continuous development of new technologies for effectively loading a target protein or peptide onto or into exosomes and effectively delivering the target protein or peptide-loaded exosomes to a target site to be treated.

Meanwhile, it is to be understood that the matters described as the background art are intended merely to aid in the understanding of the background of the present invention and are not admitted as prior art against the present invention.

### SUMMARY OF INVENTION

An object of the present invention is to provide exosomes containing an overexpressed Fc receptor or a portion thereof and a method for preparing the same.

Another object of the present invention is to provide exosomes containing an overexpressed Fc receptor or a portion thereof and a method for producing the same, wherein the exosomes are produced by transducing a genetic construct encoding a full-length Fc receptor or a portion thereof into cells that do not naturally express the Fc receptor or the portion thereof, without fusion with a nucleic acid sequence encoding an exogenous transmembrane protein or a transmembrane domain thereof.

Still another object of the present invention is to apply exosomes containing an overexpressed Fc receptor or a portion thereof to the prevention, suppression, alleviation, amelioration or treatment of immune-related diseases, cancer, inflammatory diseases, viral diseases, and various other diseases.

However, the objects of the present invention as described above are illustrative and the scope of the present invention is not limited thereby. In addition, other objects and advantages of the present invention will be more apparent from the following description, the appended claims and the accompanying drawings.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 shows a vector map of pEF6_fCD64_mGFP vector containing a nucleic acid sequence encoding a fusion polypeptide of SEQ ID NO: 2 in which mGFP is fused to the C-terminus of full-length CD64 (SEQ ID NO: 1).

FIG. 2 shows a vector map of pEF6_IgK_ecto_TM_mGFP vector containing a nucleic acid sequence encoding a fusion polypeptide of SEQ ID NO: 3 in which an immunoglobulin kappa signal peptide is fused to the N-terminus of ectodomain of CD64 and mGFP is fused to the C-terminus of ITGB1 (exogenous transmembrane protein) fused to the C-terminus of the ectodomain of CD64.

FIG. 3 shows a vector map of pEF6_IT_ecto_TM_mGFP vector containing a nucleic acid sequence encoding a fusion polypeptide of SEQ ID NO: 4 in which an ITGB1 signal peptide is fused to the N-terminus of ectodomain of CD64 and mGFP is fused to the C-terminus of ITGB1 (exogenous transmembrane protein) fused to the C-terminus of the ectodomain of CD64.

FIG. 4 depicts fluorescence micrographs showing that fluorescence is detected in HEK293T cells transfected with each of pEF6_fCD64_mGFP vector, pEF6_IgK_ecto_TM_mGFP vector, and pEF6_IT_ecto_TM_mGFP vector.

FIG. 5A shows the results of flow cytometry, indicating the content of CD64 on exosomes that were loaded with CD64 using pEF6_fCD64_mGFP vector.

FIG. 5B shows the results of flow cytometry, indicating the content of CD64 on exosomes that were loaded with CD64 using pEF6_IgK_ecto_TM_mGFP vector.

FIG. 5C shows the results of flow cytometry, indicating the content of CD64 on exosomes that were loaded with CD64 using pEF6_IT_ecto_TM_mGFP vector.

FIG. 6 is a graph showing the results of quantifying the flow cytometry results shown in FIGS. 5A to 5C as a relative value of the mean fluorescence intensity (MFI) of CD64 to the MFI of IgG isotype used as a control. FIG. 6 shows that the content of CD64 per exosome (or expression level of CD64 per exosome) is the highest when exosomes have been loaded with CD64 using a genetic construct (pEF6_fCD64_mGFP vector) encoding full-length CD64 without fusion with a nucleic acid sequence encoding an exogenous transmembrane protein or a transmembrane domain thereof.

FIG. 7 shows that exosomes containing overexpressed CD64 (fCD64 exosomes) according to the present invention are fluorescently stained with a fluorescently-labeled human anti-EGFR antibody, that is, bind to the Fc domain of the human antibody.

FIG. 8A is a standard curve showing a relationship between doxorubicin concentration and fluorescence intensity, which is used to quantify doxorubicin loaded into exosomes.

FIG. 8B is a graph showing that the absolute amount of doxorubicin loaded into exosomes increases with increasing the concentration of doxorubicin and exosomes.

FIG. 9A is a flow cytometry graph showing a rightward shift of the peak when fCD64_mGFP exosomes captured by dynabeads were incubated with an APC anti-human Fc fragment antibody, compared to a negative control (DPBS).

FIG. 9B is a flow cytometry graph showing a rightward shift of the peak when fCD64_mGFP exosomes captured by dynabeads were incubated with an APC anti-human Fc fragment antibody, compared to an antibody-untreated group.

FIG. 10A is a graph showing that when fCD64_mGFP exosomes were incubated with an APC anti-human Fc fragment antibody, while fixing the concentration of the APC anti-human Fc fragment antibody and increasing the concentration of fCD64_mGFP exosomes (number of particles/mL), MFI increased as the concentration of the exosomes increased.

FIG. 10B is a graph showing that when fCD64_mGFP exosomes were incubated with an APC anti-human Fc fragment antibody, while fixing the concentration of fCD64_mGFP exosomes (number of particles/mL) and increasing the concentration of the APC anti-human Fc fragment antibody, MFI increased as the concentration of the antibody increased.

FIG. 10C shows that when fCD64_mGFP exosomes were incubated with an APC anti-human EGFR antibody, while fixing the concentration of fCD64_mGFP exosomes (number of particles/mL) and increasing the concentration of the APC anti-human EGFR antibody, MFI increased as the concentration of the antibody increased.

FIG. 11 is a graph comparing anti-tumor or anti-cancer effects (IC₅₀ of cancer cells) of treating cancer cells with doxorubicin alone versus treating them with fCD64_mGFP exosomes loaded with the same amount of doxorubicin.

FIG. 12A depicts optical micrographs and fluorescence micrographs showing that, when MDA-MB-231 cells expressing EGFR were co-cultured with MCF-7 cells not expressing EGFR and the cells were treated with exosomes tagged with a fluorescently labeled anti-EGFR antibody, more exosomes were uptaken into MDA-MB-231 cells.

FIG. 12B is a graph comparing anti-tumor or anti-cancer effects (IC₅₀ of cancer cells) of treating cancer cells with doxorubicin-fCD64_mGFP exosomes versus treating them with doxorubicin-fCD64_mGFP exosome-anti-EGFR.

### DETAILED DESCRIPTION OF INVENTION

The present inventor has conducted extensive studies to achieve the above-described objects, and as a result, has found that, when exosomes are produced by transducing a genetic construct encoding a full-length Fc receptor or a portion thereof into cells that are unable to naturally express the Fc receptor or the portion thereof, without fusion with a nucleic acid sequence encoding an exogenous transmembrane protein or a transmembrane domain thereof, and then expressing the genetic construct in the cells, culturing the cells in a medium and isolating exosomes of interest secreted or released into the cell culture medium, the content of the Fc receptor or the portion thereof per exosome is unexpectedly high (that is, the loading efficiency of the Fc receptor or the portion thereof to the exosome is remarkably high), thereby completing the present invention.

As used herein, the term “extracellular vesicles (EVs)” is usually meant to encompass cell membrane-derived vesicles, ectosomes, shedding vesicles, microparticles, or equivalents thereto. Depending on the exosome isolation environment, conditions and method, the term “extracellular vesicles” may have the same meaning as the term “exosomes”, and may also be meant to encompass nanovesicles that have the same or similar size as exosomes but do not have the composition of exosomes. The term “exosomes” as used herein in relation to the loading of Fc receptor or portion thereof is meant to encompass the aforesaid extracellular vesicles.

As used herein, the term “exosome” refers to vesicles of tens to hundreds of nanometers in size (preferably, about 30 to 200 nm), which comprises a phospholipid bilayer membrane having the same structure as that of the cell membrane (however, the particle size of exosomes is variable depending on the type of cell from which the exosomes are isolated, an exosome isolation method and a size measurement method) (Vasiliy S. Chernyshev et al., “Size and shape characterization of hydrated and desiccated exosomes”, Anal Bioanal Chem, (2015) DOI 10.1007/s00216-015-8535-3). These exosomes contain proteins, nucleic acids (mRNA, miRNA, etc.) and the like which are called exosome cargo. It is known that exosome cargo includes a wide range of signaling factors, and these signaling factors are specific for cell types and regulated differently depending on secretory cells' environment. It is known that exosomes are intercellular signaling mediators secreted by cells, and various cellular signals transmitted through them regulate cellular behaviors, including the activation, growth, migration, differentiation, dedifferentiation, apoptosis, and necrosis of target cells.

Meanwhile, the term “exosomes” as used herein is intended to include all vesicles which are secreted from animal cells and released into extracellular spaces, and have a nano-sized vesicle structure and a composition similar to that of exosomes, e.g., exosome-like vesicles. The cells are not limited to a particular type, but as an example, not limiting the present invention, the cells may be HEK293 cells, HEK293T cells, Expi293F cells, CHO cells, stem cells, immune cells, cancer cells, or the like, and preferably may be HEK293 cells, HEK293T cells or Expi293F cells.

In addition, as an example, not limiting the present invention, the animal cells may be stem cells, immune cells, immortalized cells, or cancer cells. The stem cells may be embryonic stem cells, induced pluripotent stem cells (iPSCs), adult stem cells, embryonic stem cell-derived mesenchymal stem cells, or induced pluripotent stem cell-derived mesenchymal stem cells. The immune cells may be T cells, B cells, NK cells, cytotoxic T cells, dendritic cells, or macrophages. The adult stem cells may be at least one type of adult stem cells selected from the group consisting of mesenchymal stem cells, human tissue-derived mesenchymal stromal cells, human tissue-derived mesenchymal stem cells, and multipotent stem cells. The mesenchymal stem cells may be mesenchymal stem cells derived from at least one tissue selected from the group consisting of umbilical cord, umbilical cord blood, bone marrow, fat tissue, muscle, nerve, skin, amnion, Wharton's jelly, and placenta.

However, various animal cells that are being used in the art or may be used in the future may of course be used as long as they do not cause adverse effects on a human body. It should be noted that HEK293T cells used in the Examples described later should be understood as an example of animal cells that may be used in the present invention, and the present invention is not limited thereto.

As used herein, the term “Fc receptor” is used in a broad sense to include Fc alpha receptor (FcαR), Fe gamma receptor (FcγR), Fe epsilon receptor (FcεR), Fc mu receptor (FcμR), Fe alpha/mu receptor (Fcα/μR), FcRn (neonatal Fc receptor) and the like. As an example, not limiting the present invention, examples of Fe alpha receptor include FcαRI (CD89), examples of Fe gamma receptor include FcγRI (CD64), FcγRIIA (CD32), FcγRIIB1 (CD32), FcγRIIB2 (CD32), FcγRIIIA (CD16A) and FcγRIIIB (CD16B), and examples of Fe epsilon receptor include FcεRI and FcεRII (CD23).

The Fc alpha receptor is a receptor that binds to the Fc domain of IgA which is the most abundant immunoglobulin in a human body. CD89 is expressed on cytotoxic immune effector cells, including polymorphonuclear leukocytes (PMNs), monocytes, macrophages, neutrophils, and eosinophils. Binding of ligand to CD89 triggers phagocytosis and antibody-mediated cytotoxicity in leukocytes and CD89-bearing cell lines. CD89 can also enhance phagocytosis on target cells in concert with the receptor for IgG on effector cells.

The Fc gamma receptor is a protein that binds to an antibody portion called Fc region or Fc domain of IgG antibody and stimulates phagocytosis or cytotoxic activity via antibody-mediated phagocytosis or antibody-dependent cell-mediated cytotoxicity. For example, CD64 is a high-affinity Fe gamma receptor and is also called Fc gamma receptor I (FcγRI). The extracellular domain (ectodomain) of CD64 includes three immunoglobulin domains responsible for antibody binding. CD64 is expressed on monocytes, macrophages, dendritic cells, neutrophils, and the like, and is involved in antibody-mediated phagocytosis and cytotoxic activation. CD16 is a low-affinity Fc gamma receptor and is also called Fc gamma receptor III (FcγRIII). The extracellular domain (ectodomain) of CD16 includes two immunoglobulin domains responsible for antibody binding. CD16 exists in two different isoforms: CD16A and CD16B. CD16A is expressed on monocytes, macrophages, NK cells and the like, and induces cytotoxic activity of NK cells. CD32 is a low-affinity Fc gamma receptor and is also called Fc gamma receptor II (FcγRII). CD32 is expressed on monocytes, phagocytes, granulocytes, B cells, T cells, etc., and binds to complexed or aggregated IgG.

The Fc epsilon receptor is present on the surfaces of mast cells, basophils, eosinophils, monocytes, macrophages, and platelets. FcεRI is a high-affinity Fc epsilon receptor, and FcεRII (CD23) is a low-affinity Fc epsilon receptor. IgE primes the IgE-mediated allergic response by binding to Fc epsilon receptor present on the surfaces of mast cells and basophils.

Recycling Fc neonatal receptor (FcRn) binds to antibodies of IgG isotype and prolongs the half-life of IgG antibodies in a human body. However, FcRn does not bind to IgA and IgM isotype antibodies. FcRn is known to effectively block the degradation of IgG in lysosomes and thereby prolong the half-life of IgG.

The Fc mu receptor (FcμR) and the Fc α/μ receptor (Fcα/μR) bind antibodies of IgM isotype. Fcα/μR promotes the uptake of antibodies bound to foreign substances and immune complexes by B cells and phagocytes. In addition, FcμR is known to be important for B cell development and to affect IgM homeostasis, B cell survival, humoral immune response, and autoantibody formation.

As used herein, the term “overexpressed” or “overexpression” means that a protein or peptide that is absent or expressed at a low or normal level in wild-type cells that have not been genetically engineered and/or exosomes derived therefrom is expressed at a high level in genetically engineered cells and/or exosomes derived therefrom. In addition, the term “genetic engineering” or “genetically engineered” refers to either an action of introducing one or more genetic modifications into cells or cells made by such an action and/or exosomes derived therefrom.

As used herein, the term “transmembrane protein” refers to a protein composed of an ectodomain that is present outside a cell, a transmembrane domain that spans the cell membrane, and an endodomain that is present inside the cell. The term “exogenous transmembrane protein or transmembrane domain thereof” means any transmembrane proteins or transmembrane domains thereof, except those present in the Fc receptor loaded onto or into exosomes.

As used herein, the cells that do not naturally express an Fc receptor or a portion thereof may be, for example, HEK293 cells, HEK293T cells, or Expi293F cells. HEK293 cells, HEK293T cells, or Expi293F cells do not express an Fc receptor such as CD64, CD32, or CD16, and exosomes derived from HEK293 cells, HEK293T cells, or Expi293F cells also do not have an Fc receptors such as CD64, CD32, or CD16. The present invention is related to a technology capable of loading an Fc receptor such as CD64, CD32 or CD16 onto or into exosomes derived from HEK293 cells, HEK293T cells or Expi293F cells.

A method for producing exosomes containing an overexpressed Fc receptor or a portion thereof according to one embodiment of the present invention comprises preparing a genetic construct encoding a full-length Fc receptor or a portion thereof (provided that the portion thereof has a transmembrane protein or a transmembrane domain of the Fc receptor), without fusion with a nucleic acid sequence encoding an exogenous transmembrane protein or a transmembrane domain thereof; transducing the genetic construct into cells that do not naturally express a Fc receptor or a portion thereof; expressing the full-length Fc receptor or the portion thereof in the cells; culturing the cells in a medium; and isolating exosomes containing the full-length Fc receptor or the portion thereof.

In the method for producing exosomes containing an overexpressed Fc receptor or a portion thereof according to one embodiment of the present invention, the exosomes containing the full-length Fc receptor or the portion thereof are secreted or released from the cells and contained in the cell culture medium. The exosomes containing the full-length Fc receptor or the portion thereof may be isolated from the cell culture medium.

In the method for producing exosomes containing an overexpressed Fc receptor or a portion thereof according to one embodiment of the present invention, the full-length Fc receptor or the portion thereof is located on a surface of the exosomes. In the present invention, the full-length Fc receptor or the portion thereof is displayed on the surface of the exosomes by its own transmembrane protein or transmembrane domain thereof that is present in the full-length Fc receptor or the portion thereof.

In the method for producing exosomes containing an overexpressed Fc receptor or a portion thereof according to one embodiment of the present invention, the Fc receptor may be CD64, CD32 or CD16. In addition, the cells that do not naturally express the Fc receptor or the portion thereof may be HEK293 cells, HEK293T cells, or Expi293F cells.

As an example, not limiting the present invention, the Fc receptor may be FcαRI (CD89), FcγRI (CD64), FcγRIIA (CD32), FcγRIIB1 (CD32), FcγRIIIB2 (CD32), FcγRIIIA (CD16A), FcγRIIIB (CD16B), FcεRI, FcεRII (CD23), FcμR, Fcα/μR, or FcRn.

In addition, the present invention provides exosomes containing an overexpressed Fc receptor or a portion thereof, produced according to the above-described method, and provides a composition comprising, as an active ingredient, the exosomes containing the overexpressed Fc receptor or the portion thereof. The composition may be a pharmaceutical composition or a cosmetic composition. For example, the pharmaceutical composition may be prepared as an injectable formulation.

The pharmaceutical composition according to one embodiment of the present invention comprises the exosomes containing the overexpressed Fc receptor or the portion thereof, and a pharmaceutically acceptable carrier. For example, the pharmaceutical composition may be used for enhancing an anti-tumor effect or an anti-cancer effect.

The pharmaceutical composition according to one embodiment of the present invention may comprise a cancer cell killing agent. The cancer cell killing agent may be located on the surface of the exosomes or inside the exosomes, or may also be fused to the overexpressed Fc receptor or the portion thereof.

For example, the cancer cell killing agent may be at least one selected from the group consisting of cytokines, including IL-2, IL-7, IL-12, IL-15, IL-18, and IL-21, toxin proteins, DNA damage-inducing proteins, CRISPR-binding proteins, apoptosis-inducing proteins, granzyme A, granzyme B, perforin, FAS protein, TRAIL (TNF-related apoptosis-inducing ligand) protein, antibodies against immune checkpoint proteins such as PD-1, PD-L1, and CTLA-4, cancer-specific antibodies such as anti-CD47 antibody and anti-EGFR antibody, immune cell surface proteins such as T cell receptor and NKG2D, stimulator of interferon genes (STING), STING agonists, albumin-bound paclitaxel, Actinomycin, Alitretinoin, Azacitidine, Azathioprine, Bevacizumab, Bexatotene, Bleomycin, Bortezomib, Carboplatin, Capecitabine, Cetuximab, Cisplatin, Chlorambucil, Cyclophosphamide, Cytarabine, Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Ipilimumab, Irinotecan, Lapatinib, Mechlorethamine, Melphalan, Mercaptopurine, Methotrexate, Mitoxantrone, Ocrelizumab, Ofatumumab, Oxaliplatin, Paclitaxel, Panitumab, Pemetrexed, Rituximab, Tafluposide, Teniposide, Tioguanine, Topotecan, Tretinoin, Valrubicin, Vemurafenib, Vinblastine, Vincristine, Vindesine, Vinorelbine, Vorinostat, Romidepsin, 5-fluorouracil (5-FU), 6-mercaptopurine (6-MP), Cladribine, Clofarabine, Floxuridine, Fludarabine, Pentostatin, Mitomycin, Ixabepilone, Estramustine, and the like. However, the present invention is not limited thereto, and it is of course possible to use various anti-tumor or anti-cancer agents known in the art.

Additionally, the present invention provides a drug delivery system comprising the pharmaceutical composition described above.

The drug delivery system according to one embodiment of the present invention may further comprise an antibody. The antibody may be co-administered with the exosomes.

In the pharmaceutical composition or drug delivery system according to one embodiment of the present invention, the antibody may be at least one selected from the group consisting of 3F8, 8H9, Abagovomab, Avelumab, Abciximab, Actoxumab, Adalimumab, Adecatumumab, Aducanumab, Afelimomab, Afutuzumab, Alacizumab pegol, ALD518, Alemtuzumab, Alirocumab, Altumomab pentetate, Amatuximab, Anatumomab mafenatox, Anifrolumab, Anrukinzumab, Apolizumab, Arcitumomab, Aselizumab, Atinumab, Atlizumab, Atorolimumab, Bapineuzumab, Basiliximab, Bavituximab, Bectumomab, Belimumab, Benralizumab, Bertilimumab, Besilesomab, Bevacizumab, Bezlotoxumab, Biciromab, Bimagrumab, Bivatuzumab mertansine, Blinatumomab, Blosozumab, Brentuximab vedotin, Briakinumab, Brodalumab, Canakinumab, Cantuzumab mertansine, Cantuzumab ravtansine, Caplacizumab, Capromab pendetide, Carlumab, Catumaxomab, CC49, cBR96-doxorubicin immunoconjugate, Cedelizumab, Certolizumab pegol, Cetuximab, Ch.14.18, Citatuzumab bogatox, Cixutumumab, Clazakizumab, Clenoliximab, Clivatuzumab tetraxetan, Conatumumab, Concizumab, Crenezumab, CR6261, Dacetuzumab, Daclizumab, Dalotuzumab, Daratumumab, Demcizumab, Denosumab, Detumomab, Dorlimomab aritox, Drozitumab, Duligotumab, Dupilumab, Dusigitumab, Ecromeximab, Eculizumab, Edobacomab, Edrecolomab, Efalizumab, Efungumab, Eldelumab, Elotuzumab, Elsilimomab, Enavatuzumab, Enlimomab pegol, Enokizumab, Enoticumab, Ensituximab, Epitumomab cituxetan, Epratuzumab, Erlizumab, Ertumaxomab, Etaracizumab, Etrolizumab, Evolocumab, Exbivirumab, Fanolesomab, Faralimomab, Farletuzumab, Fasinumab, FBTA05, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Igovomab, Ipilimumab, IMAB362, Imciromab, Imgatuzumab, Inclacumab, Indatuximab ravtansine, Infliximab, Intetumumab, Inolimomab, Inotuzumab ozogamicin, Iratumumab, Itolizumab, Ixekizumab, Keliximab, Labetuzumab, Lambrolizumab, Lampalizumab, Lebrikizumab, Lemalesomab, Lerdelimumab, Lexatumumab, Libivirumab, Ligelizumab, Lintuzumab, Lirilumab, Lodelcizumab, Lorvotuzumab mertansine, Lucatumumab, Lumiliximab, Mapatumumab, Margetuximab, Maslimomab, Mavrilimumab, Matuzumab, Mepolizumab, Metelimumab, Milatuzumab, Minretumomab, Mitumomab, Mogamulizumab, Morolimumab Motavizumab, Moxetumomab pasudotox, Muromonab-CD3, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Ocaratuzumab, Ocrelizumab, Odulimomab, Ofatumumab, Olaratumab, Olokizumab, Omalizumab, Onartuzumab, Ontuxizumab, Oportuzumab monatox, Oregovomab, Orticumab, Otelixizumab, Otlertuzumab, Oxelumab, Ozanezumab, Ozoralizumab, Pagibaximab, Palivizumab, Panitumumab, Pankomab, Panobacumab, Parsatuzumab, Pascolizumab, Pateclizumab, Patritumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, PRO 140, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, SGN-CD19A, SGNCD33A, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Teneliximab, Teplizumab, Teprotumumab, TGN1412, Ticilimumab, Tildrakizumab, Tigatuzumab, TNX-650, Tocilizumab, Toralizumab, Tositumomab, Bexxar, Tovetumab, Tralokinumab, Trastuzumab, TRBSO7, Tregalizumab, Tremelimumab, Tucotuzumab celmoleukin, Tuvirumab, Ublituximab, Urelumab, Urtoxazumab, Ustekinumab, Vantictumab, Vapaliximab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab, and Zolimomab aritox. However, the present invention is not limited thereto, and it is of course possible to use various antibodies known in the art.

As an example, not limiting the present invention, the pharmaceutical composition or drug delivery system according to one embodiment of the present invention may be any formulation for oral or parenteral administration

The pharmaceutical composition or drug delivery system according to one embodiment of the present invention may be used to prevent, suppress, alleviate, ameliorate or treat immune-related diseases, cancer, inflammatory diseases, viral diseases, and/or other various diseases.

The pharmaceutical composition or drug delivery system according to one embodiment of the present invention may comprise pharmaceutically acceptable carriers, excipients, diluents or the like. The carriers, excipients and dilutes include, but are not limited to, lactose, dextrose, trehalose, sucrose, sorbitol, mannitol, xylitol, erythritol, maltitol, starch, acacia gum, alginate, gelatin, calcium phosphate, calcium carbonate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil. For use, the pharmaceutical composition or drug delivery system according to one embodiment of the present invention may be formulated as oral dosage forms, such as powders, pills, tablets, capsules, suspensions, emulsions, syrups, granules, elixirs, aerosols, or the like, skin external preparations, suppositories, or sterile injectable solutions, according to conventional methods.

Administration of the pharmaceutical composition or drug delivery system according to one embodiment of the present invention means introducing a desired substance into a patient by any appropriate method, and the pharmaceutical composition or drug delivery system may be administered by any general route, as long as the drug can reach a target tissue. For example, the pharmaceutical composition or drug delivery system according to one embodiment of the present invention may be administered orally or parenterally. Routes for parenteral administration may include intratumoral administration, intra-articular administration, intrasynovial administration, intrasternal administration, intrathecal administration, intralesional administration, intracranial administration, transdermal administration, intraperitoneal administration, intravenous administration, intra-arterial administration, intra-lymphatic administration, intramuscular administration, subcutaneous administration, intradermal administration, topical administration, intrarectal administration, and the like. However, the scope of the present invention is not limited thereto, and various administration methods known in the art are not excluded. Furthermore, the pharmaceutical composition or drug delivery system according to one embodiment may be administered by any device through which an active ingredient may be delivered into a target tissue or cell. In addition, the effective amount of the pharmaceutical composition or drug delivery system according to one embodiment of the present invention means the amount required for administration in order to achieve the effect of treating a disease.

A solid formulation for oral administration of the pharmaceutical composition or drug delivery system according to one embodiment of the present invention may be prepared by mixing at least one excipient, for example, starch, calcium carbonate, sucrose, lactose, gelatin, and the like. In addition, the solid formulation for oral administration may comprise a lubricant such as silica, stearic acid, magnesium stearate, calcium stearate, talc, or polyethylene glycol, in addition to the above excipient. A liquid formulation for oral administration of the pharmaceutical composition or drug delivery system according to one embodiment of the present invention may comprise various excipients, such as wetting agents, sweeteners, aromatics, and preservatives, in addition to simple diluents such as water and liquid paraffin.

Formulations for parenteral administration of the pharmaceutical composition or drug delivery system according to the present invention may be sterilized aqueous solutions, non-aqueous solutions, suspensions, emulsions, lyophilized formulations, or suppositories. Formulations for parenteral administration of the pharmaceutical composition or drug delivery system according to one embodiment of the present invention may also be prepared as injectable formulations. Injectable formulations according to one embodiment of the present invention may be aqueous injectable formulations, non-aqueous injectable formulations, aqueous suspension injections, non-aqueous suspension injections, solid injectable formulations which are used after dissolution or suspension, etc., but are not limited thereto. An injectable formulation according to one embodiment of the present invention may further comprise at least one of distilled water for injection, vegetable oils (e.g., peanut oil, sesame oil, camellia oil, etc.), monoglyceride, diglyceride, propylene glycol, camphor, estradiol benzoate, bismuth subsalicylate, arsenobenzol sodium, or streptomycin sulfate, depending on the type thereof, and may optionally further comprise a stabilizer or a preservative.

The content of the pharmaceutical composition or drug delivery system according to one embodiment in a formulation may be suitably selected depending on the kind, amount, form and the like of additional components as described above. For example, the pharmaceutical composition or drug delivery system of the present invention may be contained in an amount of about 0.1 to 99 wt %, preferably about 10 to 90 wt %, based on the total weight of an injectable formulation. Furthermore, the suitable dose of the pharmaceutical composition or drug delivery system according to one embodiment of the present invention may be adjusted depending on the kind of patient's disease, the severity of the disease, the type of formulation, formulating method, patient's age, sex, body weight, health condition, diet, excretion rate, the period of administration, and the regime of administration. For example, when the pharmaceutical composition or drug delivery system according to one embodiment of the present invention is administered to an adult, it may be administered once to several times at a dose of 0.001 mg/kg to 100 mg/kg per day.

Meanwhile, when the composition according to one embodiment of the present invention is prepared as a cosmetic composition, it may suitably contain components which are generally used in cosmetic products, for example, moisturizers, antioxidants, oily components, UV absorbers, emulsifiers, surfactants, thickeners, alcohols, powder components, colorants, aqueous components, water, and various skin nutrients, etc., as needed, within the range that does not impair the effect of the present invention.

The cosmetic composition according to one embodiment of the present invention may be used in various forms, for example, a patch, a mask pack, a mask sheet, a cream, a tonic, an ointment, a suspension, an emulsion, a paste, a lotion, a gel, an oil, a pack, a spray, an aerosol, a mist, a foundation, a powder and an oilpaper.

The cosmetic composition according to one embodiment of the present invention may be prepared as any cosmetic formulation which is generally prepared in the art. For example, it may be formulated as a patch, a mask pack, a mask sheet, a skin softener, a nutrition, an astringent lotion, a nourishing cream, a massage cream, an eye cream, a cleansing cream, an essence, an eye essence, a cleansing lotion, a cleansing foam, a cleansing water, a sunscreen, a lipstick, a soap, a shampoo, a surfactant-containing cleanser, a bath preparation, a body lotion, a body cream, a body oil, a body essence, a body cleanser, a hairdye, a hair tonic, etc., but is not limited thereto.

The cosmetic composition according to one embodiment of the present invention contains components which are commonly used in cosmetic products. For example, the cosmetic composition may contain conventional adjuvants and carriers, such as antioxidants, stabilizers, solubilizers, vitamins, pigments, and fragrances. In addition, other components in each formulation for the cosmetic composition may be suitably selected without difficulty by those skilled in the art depending on the type or intended use of cosmetic composition.

### Advantageous Effects

The present invention has the advantage that when exosomes are produced using a genetic construct encoding a full-length Fc receptor or a portion thereof without fusion with a nucleic acid sequence encoding an exogenous transmembrane protein or a transmembrane domain thereof, it is possible to load the Fc receptor or the portion thereof onto or into exosomes at a higher density, compared to a conventional method, which involves loading an Fc receptor or a portion thereof onto or into exosomes by fusing it to an exogenous transmembrane protein or a transmembrane domain thereof. That is, the method for producing exosomes containing an overexpressed Fc receptor or a portion, according to the present invention, has the advantage of being able to load the Fc receptor or the portion thereof onto or into exosomes at a higher density with higher efficiency, compared to a conventional method, which involves loading an Fc receptor or a portion thereof onto or into exosomes by fusing it to an exogenous transmembrane protein or a transmembrane domain thereof.

It should be understood that the scope of the present invention is not limited to the aforementioned effects.

### Examples

Hereinafter, the present invention will be described in more detail with reference to the following examples. However, the following examples are only to illustrate the present invention and are not intended to limit or restrict the scope of the present invention. Those that can be easily inferred by those skilled in the art from the detailed description and examples of the present invention are interpreted as falling within the scope of the present invention. References referred to in the present invention are incorporated herein by reference.

Throughout the present specification, it is to be understood that, when any part is referred to as “comprising” any component, it does not exclude other components, but may further include other components, unless otherwise specified.

### Example 1: Preparation of Vectors Expressing Fusion Polypeptide of Interest

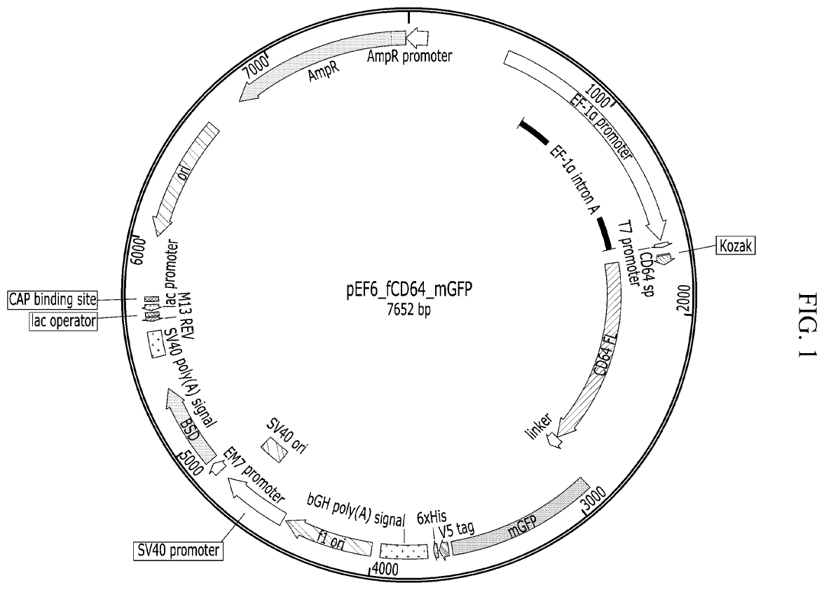

The KpnI and XbaI sites in the multicloning region of pEF6/V5-His A vector (#V96120, purchased from Invitrogen, USA) were respectively digested with KpnI and XbaI restriction enzymes to linearize the vector's DNA. DNA fragments encoding fusion polypeptides corresponding to SEQ ID NO: 2, SEQ ID NO: 3, and SEQ ID NO: 4, respectively, were amplified by PCR, and then each of the amplified fragments was subcloned into pEF6/V5-His A vector (see FIGS. 1 to 3).

A linker (GGGGS) was repeatedly inserted four times between full-length CD64 (SEQ ID NO: 1) and mGFP, which constitute the fusion polypeptide having the amino acid sequence of SEQ ID NO: 2. In addition, the linker (GGGGS) was repeatedly inserted four times between the ectodomain of CD64 and the exogenous transmembrane protein (ITGB1) in the fusion polypeptide having the amino acid sequence of SEQ ID NO: 3 or SEQ ID NO: 4. The same linker was also repeatedly inserted four times between the exogenous transmembrane protein (ITGB1) and mGFP.

FIG. 1 shows a vector that contains a nucleic acid sequence encoding a fusion polypeptide (fusion polypeptide having the amino acid sequence of SEQ ID NO: 2) in which mGFP is fused to the C-terminus of full-length CD64 (hereinafter referred to as “fCD64”). The vector shown in FIG. 1 is hereinafter referred to as “pEF6_fCD64_mGFP”.

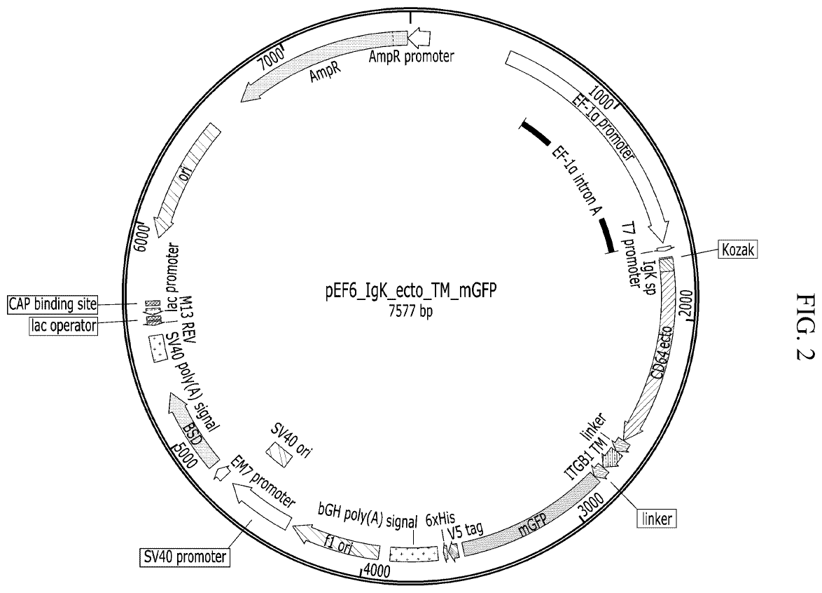

In addition, FIG. 2 shows a vector that contains a nucleic acid sequence encoding a fusion polypeptide (fusion polypeptide having the amino acid sequence of SEQ ID NO: 3) in which an immunoglobulin kappa signal peptide (hereinafter referred to as “IgK”) is fused to the N-terminus of the ectodomain (hereinafter referred to as “ecto”) of CD64 and mGFP is fused to the C-terminus of the exogenous transmembrane protein ITGB1 (hereinafter referred to as “TM”) fused to the C-terminus of the ectodomain of CD64. The vector shown in FIG. 2 is hereinafter referred to as “pEF6_IgK_ecto_TM_mGFP”.

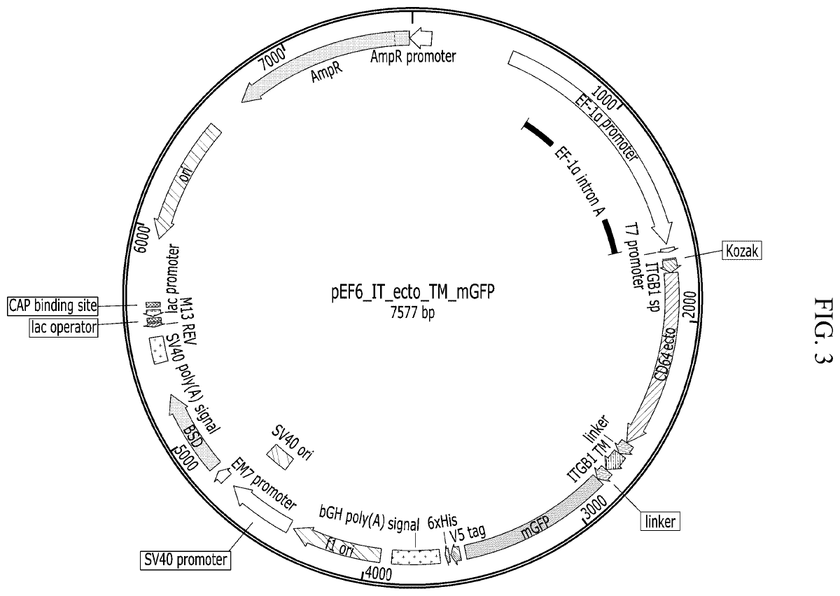

Further, FIG. 3 shows a vector that contains a nucleic acid sequence encoding a fusion polypeptide (fusion polypeptide having the amino acid sequence of SEQ ID NO: 4) in which the signal peptide of ITGB1 (hereinafter referred to as “IT”) is fused to the N-terminus of the ectodomain of CD64 and mGFP is fused to the C-terminus of the exogenous transmembrane protein ITGB1 fused to the C-terminus of the ectodomain of CD64. The vector shown in FIG. 3 is hereinafter referred to as “pEF6_IT_ecto_TM_mGFP”.

### Example 2: Culture of Cells and Establishment of Stable Cell Lines

HEK293T cells (purchased from Horizon Discovery) to be used for transduction were subcultured in DMEM (purchased from ThermoFisher Scientific, USA) containing 10% fetal bovine serum (FBS; purchased form ThermoFisher Scientific, USA) and 1% antibiotics-antimycotics (purchased from ThermoFisher Scientific, USA) at 37° C. under 5% CO₂, according to the supplier's recommendations.

To establish cell lines stably expressing each of the fusion polypeptides of interest prepared in Example 1, each of pEF6_fCD64_mGFP vector, pEF6_IgK_ecto_TM_mGFP vector, and pEF6_IT_ecto_TM_mGFP vector prepared in Example 1 was transducing into HEK293T cells using Effectene transfection reagent (purchased from Qiagen, Germany) and cultured for 48 hours after the transduction. The cells were cultured in the above-described DMEM supplemented with 10 μg/mL Blasticidin (purchased from InvivoGen, USA) for 3 weeks, and only cells stably expressing each of the fusion polypeptides of interest were selected.

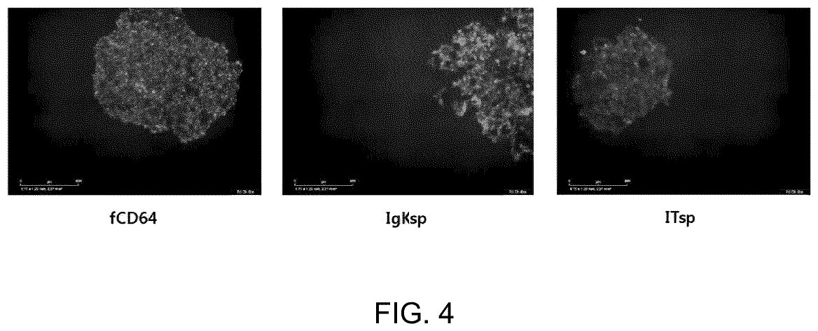

Thereafter, it was checked that each of the fusion polypeptides of interest was expressed in the HEK293T cells that were selected as described above, using Incucyte S3 (purchased from Sartorius, Germany). Since all of the three vectors prepared in Example 1 contain the mGFP gene, fluorescence should be detected in the HEK293T cells when each of these vectors was normally transduced into the HEK293T cells and stably expressed in the HEK293T cells. As a result of taking images with a fluorescence microscope after 7 days of culturing, fluorescence was detected in the HEK293T cells that were transduced with each of pEF6_fCD64_mGFP vector, pEF6_IgK_ecto_TM_mGFP vector and pEF6_IT_ecto_TM_mGFP vector (see FIG. 4). Accordingly, it can be seen that each of the three vectors prepared in Example 1 was normally transduced into the HEK293T cells, and each fusion polypeptide of interest encoded by each of these vectors was normally expressed in the HEK293T cells.

### Example 3: Exosome Isolation and CD64 Quantification

The HEK293T cell lines stably expressing the fusion polypeptides of SEQ ID NO: 2, SEQ ID NO: 3, and SEQ ID NO: 4, respectively, as prepared in Example 2, were dispensed in a 75T cell culture flask at a density of 3.75×10⁶ cells and cultured at 37° C. under 5% CO₂. The next day, the medium was replaced with Opti-MEM (purchased from ThermoFisher Scientific, USA) containing 1% exosome-depleted FBS (purchased from System Biosciences, USA), 1% antibiotics-antimycotics and 10 μg/mL Blasticidin (purchased from InvivoGen, USA), and the cells were cultured for 48 hours at 37° C. under 5% CO₂.

Next, the cell culture supernatant was taken and filtered through a 0.22-am syringe filter (product name: SLGVR33RB; purchased from Merck, Germany). 5 mL of the filtered supernatant was concentrated at 4° C. for 1 hour using Amicon Ultra-15 Centrifugal 100 kDa Filter Unit (purchased from Merck, Germany). The concentrate was suspended in DPBS (Dulbecco's Phosphate-Buffered Saline; purchased from Gibco, USA). PEG8000 (500 mg/mL) solution was added to the suspended concentrate in an amount corresponding to 20% of the total volume. The concentrate and PEG8000 solution were mixed, and the resulting mixture was stored at 4° C. After 24 hours, the concentrate mixed with PEG8000 was centrifuged at 10,000×g at 4° C., and then the supernatant was completely removed to obtain an exosome pellet. The exosome pellet was suspended in 200 μL DPBS, and the protein concentration therein was quantified using a microBCA assay kit (purchased from ThermoFisher Scientific, USA). Thereafter, the exosomes were adjusted to a concentration of 100 μg/mL by adding DPBS thereto and stored at −80° C. until use.

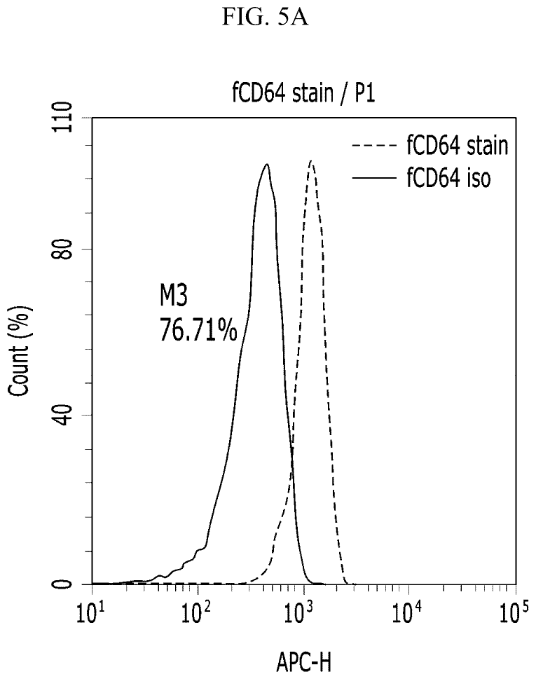

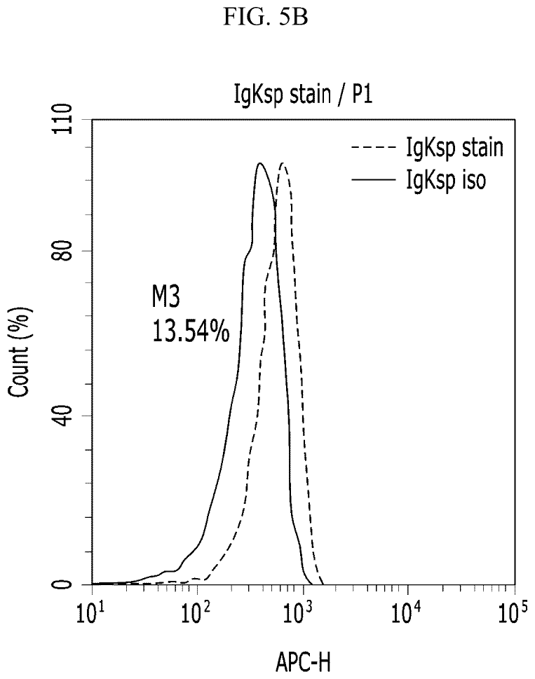

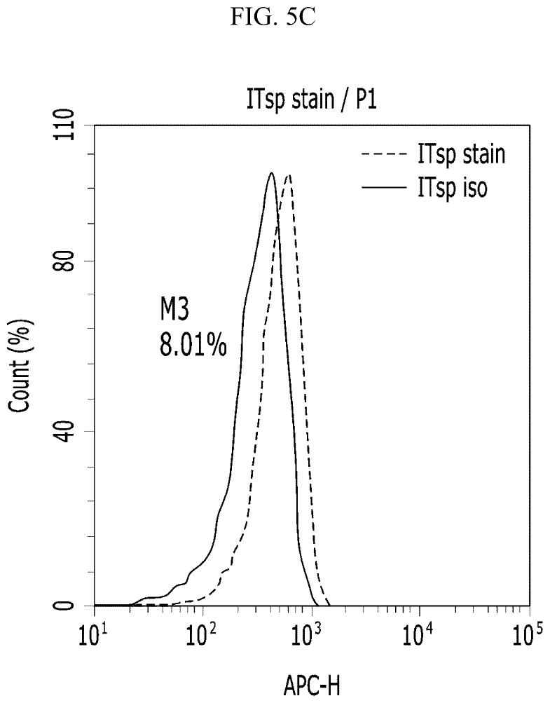

In order to determine the content (or expression level) of CD64 on the surface of each exosome, the exosomes were captured using CD81 Dynabeads (purchased from ThermoFisher Scientific, USA). This is a method of capturing only exosomes using 2.7 m beads since the size of exosomes is so small that it is impossible to analyze them by a general flow cytometer. The exosomes were stained with “APC mouse IgG1, K isotype control antibody” or “APC anti-human CD64 antibody” (purchased from BioLegend, USA) at room temperature for 1 hour. Then, flow cytometry was performed using a Novocyte 2000R flow cytometer (purchased from Agilent, USA) (FIGS. 5A to 5C). FIG. 5A shows the results of flow cytometry, indicating the content of CD64 on exosomes that were loaded with CD64 using pEF6_fCD64_mGFP vector. FIG. 5B shows the results of flow cytometry, indicating the content of CD64 on exosomes that were loaded with CD64 using pEF6_IgK_ecto_TM_mGFP vector. FIG. 5C shows the results of flow cytometry, indicating the content of CD64 on exosomes that were loaded with CD64 using pEF6_IT_ecto_TM_mGFP vector.

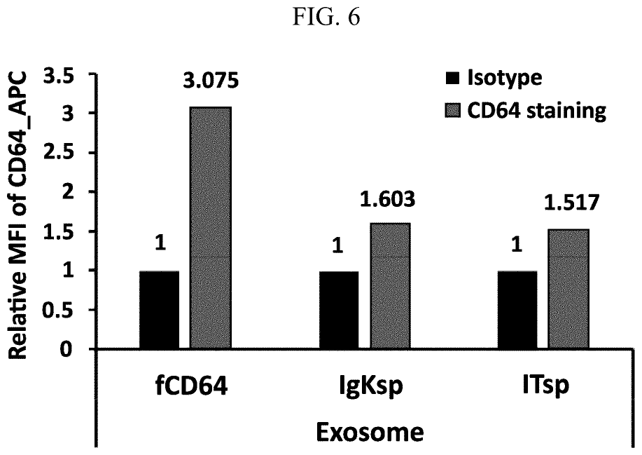

The flow cytometry results in FIGS. 5A to 5C were quantified as a relative value of the mean fluorescence intensity (MFI) of CD64 to the MFI of IgG isotype used as a control. As shown in FIG. 6, the CD64 content (or expression level) on the exosome surface was approximately two times higher in the exosomes isolated from the culture medium of the cell line stably expressing pEF6_fCD64_mGFP, compared to the exosomes isolated from the culture medium of the cell line stably expressing pEF6_IgK_ecto_TM_mGFP or pEF6_IT_ecto_TM_mGFP. These results indicate that the CD64 content (or expression level) per exosome was unexpectedly highest when exosomes were loaded with CD64 using the genetic construct (pEF6_fCD64_mGFP vector) that encodes full-length CD64, without fusion with a nucleic acid sequence encoding an exogenous transmembrane protein or a transmembrane domain thereof, according to the present invention.

### Example 4: Evaluation of Binding Affinity of Exosomes to Fc Domain

CD64, which is expressed on the surfaces of immune cells such as macrophages and dendritic cells, binds to the Fc domain of an antibody's constant region. This allows immune cells to recognize the antibody bound to the antigen, causing an antigen-specific immune response.

In addition to the CD64 loading onto exosomes as confirmed in Example 3, the present inventor examined whether CD64 loaded onto the exosomes would exhibit an antibody-binding function. The exosomes isolated from the culture medium of the non-transformed HEK293T cell line and the exosomes isolated from the culture medium of the HEK293T cell line stably expressing pEF6_fCD64_mGFP (hereinafter referred to as “fCD64_mGFP exosomes”), were captured with CD81 Dynabeads, respectively. Thereafter, the exosomes were stained with human EGFR Alexa Fluor 488-conjugated antibody (purchased from R&D Systems, USA) at room temperature for 1 hour, and then analyzed with a Novocyte 2000R flow cytometer.

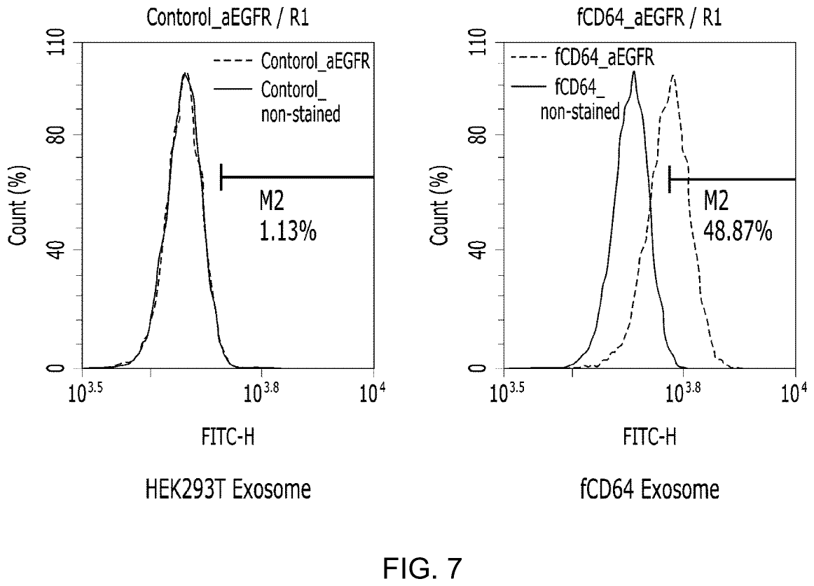

As shown in FIG. 7, there was no difference in FITC fluorescence intensity between when the exosomes isolated from the culture medium of the non-transformed HEK293T cells were stained with the antibody and when they were not stained (shown in the left graph in FIG. 7). However, when the fCD64_mGFP exosomes were stained with the antibody, about 48.9% of the exosomes bound to the antibody, indicating an increased FITC fluorescence intensity (shown in the right graph in FIG. 7). This indicates that the fCD64_mGFP exosomes are capable of binding to the Fc domain of a human antibody.

Meanwhile, antibody-based targeted anticancer agents bind to a cancer cell-specific antigen and exhibit antitumor or anticancer effects. The exosomes containing an overexpressed Fc receptor such as CD64, CD32 or CD16 or a portion thereof, according to the present invention, have a higher density of the Fc receptor or the portion thereof on their surface compared to conventional exosomes. The exosomes containing an overexpressed Fc receptor or a portion thereof on their surface, according to the present invention, are more chemotactic to the Fc domain of an antibody, and the Fc receptor or the portion thereof on the exosomes binds specifically to the Fc domain of the antibody. Therefore, when the exosomes containing an overexpressed Fc receptor or a portion thereof and loaded with a cancer cell killing agent are administered to a cancer patient, the exosomes can allow for an additional and specific attack on cancer cells against which an antibody anticancer agent with an Fc domain acts, due to the Fc receptor or the portion thereof present on the exosome surface at a higher density, thereby increasing antitumor or anticancer effects.

### Example 5: Preparation of Exosomes Loaded with Doxorubicin

When a chemotherapeutic drug, such as doxorubicin (DOX), which is a toxin commonly used in the treatment of solid tumors and various cancers, is loaded into the exosomes and delivered to target tumor cells, the anti-tumor efficacy thereof can be enhanced. Doxorubicin can be loaded into the exosomes by incubating the same with the exosomes at room temperature. Through the process described below, 20.4±0.5 wt % of doxorubicin out of 100 wt % of total doxorubicin introduced was finally loaded into the exosomes.

To load doxorubicin into the exosomes, 1,000 μg of fCD64_mGFP exosomes were mixed with 2,000 μg/mL doxorubicin hydrochloride overnight at room temperature. Since doxorubicin has a characteristic of forming precipitates over time when mixed with various neutral buffers, doxorubicin and the exosomes were mixed using a hulamixer sample mixer (purchased from Thermofisher, USA) at various angles overnight in order to prevent the precipitate formation. Thereafter, doxorubicin that was not loaded into the exosomes was removed by ultra-high-speed centrifugation. The exosomes loaded with doxorubicin were used after mixing with DPBS buffer filtered through a 0.1-μm syringe filter (product name: SLVVR33RS; purchased from Merck, Germany).

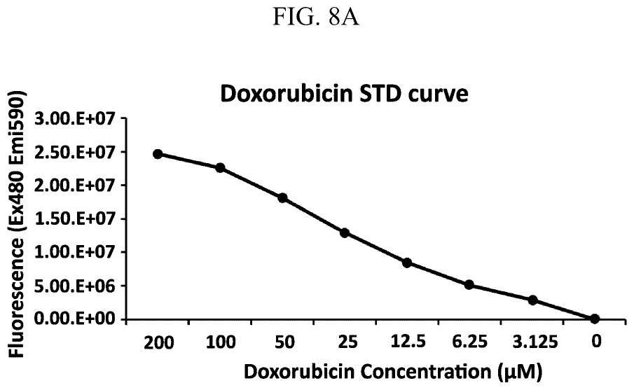

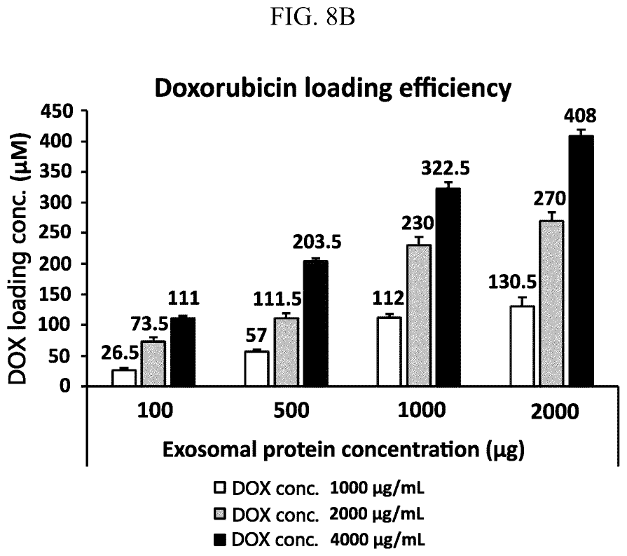

The amount of doxorubicin loaded into the exosomes was measured by analyzing the fluorescence intensity of doxorubicin (excitation wavelength: 480 nm, and emission wavelength: 590 nm). Unloaded doxorubicin was also analyzed and measured in the same manner. A standard curve between the known concentration of doxorubicin and the measured fluorescence intensity of doxorubicin was created (FIG. 8A), and the concentration of doxorubicin loaded into the exosomes was measured using this standard curve (FIG. 8B). As shown in FIG. 8B, it can be seen that the absolute amount of doxorubicin loaded into the exosomes increases with increasing the concentration of doxorubicin and exosomes.

### Example 6: Evaluation of Antibody's Binding Affinity to fCD64_mGFP Exosomes

In this Example, exosomes containing overexpressed CD64 were produced, and then it was confirmed that CD64 was present on the surface of the produced exosome, and that an antibody actually bound to the surface of the exosome. CD64-loaded exosomes (fCD64_mGFP exosomes) produced according to Examples 1 to 3 were collected in large quantities and pooled, thus preparing high-concentration exosomes. Then, the exosomes were captured using CD81 Dynabeads (purchased from ThermoFisher Scientific, USA), and the antibody's binding affinity to the surface of the captured exosomes was evaluated.

The fCD64_mGFP exosomes were incubated and stained with each of “APC mouse IgG1, x isotype control antibody”, “APC anti-human EGFR antibody (purchased from R&D Bioscience, USA)” and “APC anti-human Fc fragment antibody (purchased from Jackson ImmunoResearch, USA)” for 1 hour at room temperature. Then, flow cytometry was performed using a Novocyte 2000R flow cytometer (purchased from Agilent, USA).

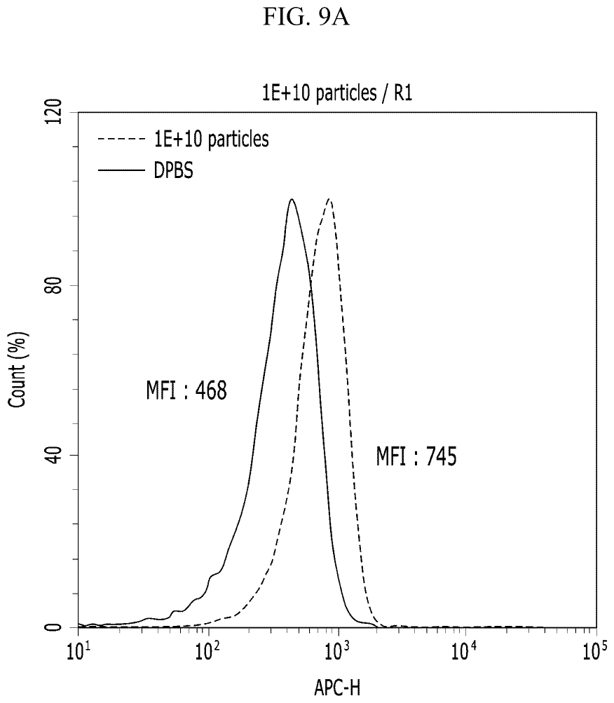

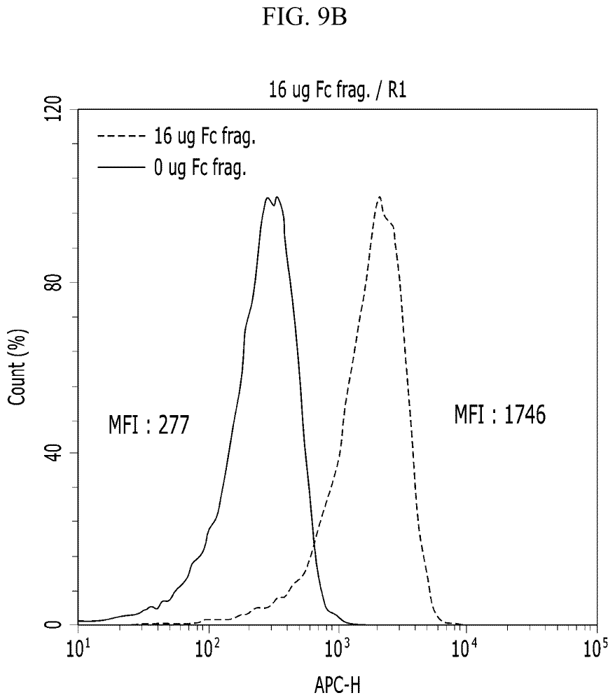

As shown in FIGS. 9A and 9B, it was confirmed that the peak shifted rightward when fCD64_mGFP exosomes captured by dynabeads were incubated with the APC anti-human Fc fragment antibody, compared to a negative control (DPBS) and an antibody-untreated group. These results indicate that CD64 artificially displayed on the fCD64_mGFP exosomes normally bound to the Fc domain of the APC-labeled antibody, and that the fCD64_mGFP exosomes were labeled by APC through binding of CD64 to the Fc domain.

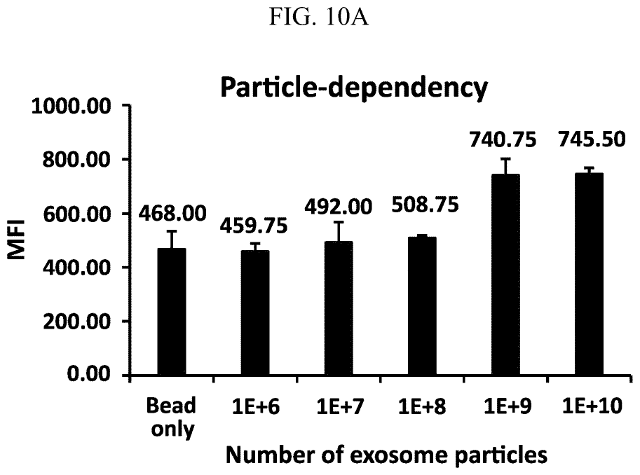

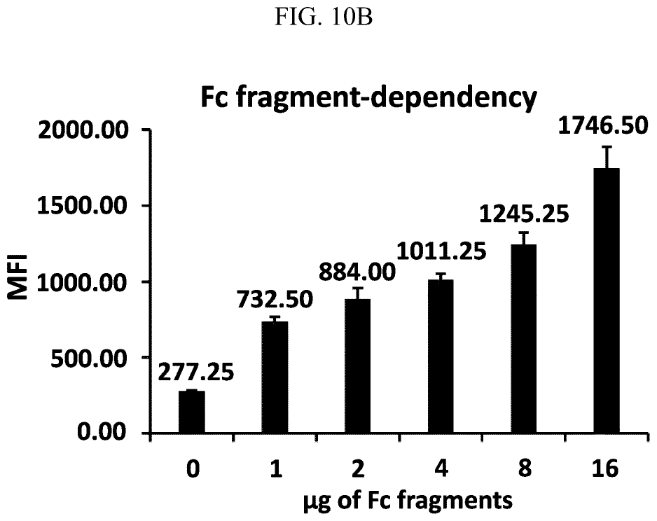

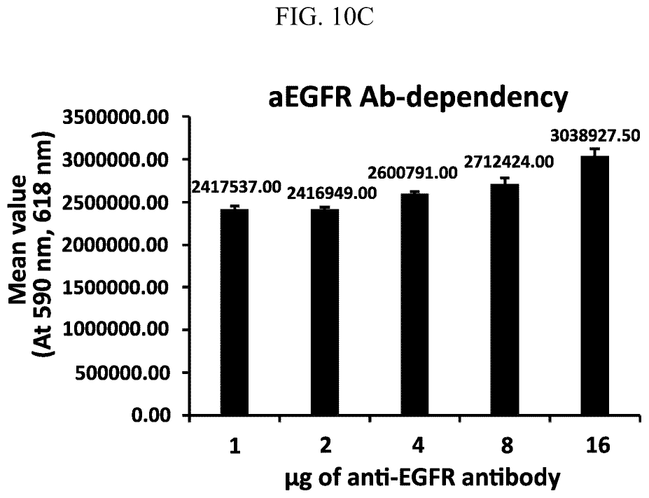

Meanwhile, fCD64_mGFP exosomes were incubated with the APC anti-human Fc fragment antibody, while fixing the concentration of the APC anti-human Fc fragment antibody at 2 μg/mL and increasing the concentration of the fCD64_mGFP exosomes (number of particles/mL), and then flow cytometry was performed using a Novocyte 2000R flow cytometer. As a result, it was confirmed that the mean fluorescence intensity (MFI) from APC increased as the exosome concentration increased (see FIG. 10A).

In addition, fCD64_mGFP exosomes were incubated with the APC anti-human Fc fragment antibody or the APC anti-human EGFR antibody, while fixing the concentration of the fCD64_mGFP exosomes (number of particles/mL) at 1×10⁹ particles/mL and increasing the concentration of the APC anti-human Fc fragment antibody or the APC anti-human EGFR antibody, and then flow cytometry was performed using a Novocyte 2000R flow cytometer. As a result, it was confirmed that the mean fluorescence intensity (MFI) from APC increased as the concentration of the antibody increased (see FIGS. 10B and 10C).

The above results mean that the antibody's binding affinity to the fCD64_mGFP exosomes increases in a manner dependent on the concentration of the antibody. In addition, the above results mean that the binding affinity of the antibody with the Fc domain increases in a concentration-dependent manner since CD64 is present at a high density on the surface of the fCD64_mGFP exosomes.

### Example 7: Anti-Tumor Efficacy of Exosomes Loaded with Doxorubicin

### Example 7-1: Comparison of Antitumor or Anticancer Efficacy Between Doxorubicin Alone and Doxorubicin-fCD64_mGFP Exosomes

In this Example, the anti-tumor or anti-cancer efficacy of fCD64_mGFP exosomes loaded with doxorubicin was examined as follows.

The MDA-MB-231 human breast cancer cell line (10,000 cells/well) was treated in vitro with doxorubicin alone or the fCD64_mGFP exosomes loaded with the same amount of doxorubicin (hereinafter referred to as “doxorubicin-fCD64_mGFP exosomes”). The breast cancer cells were treated with doxorubicin-fCD64_mGFP exosomes at a concentration of 2.56×10¹¹ particles/mL.

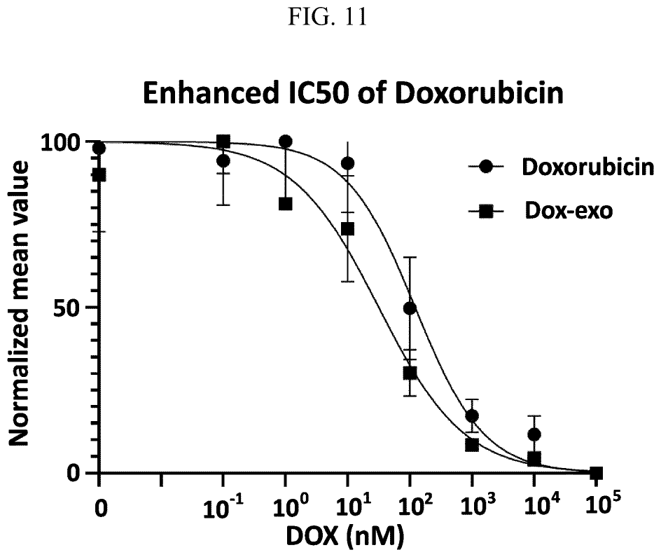

As a result, it was confirmed that both treatment with doxorubicin alone and treatment with doxorubicin-fCD64_mGFP exosomes inhibited the growth of the breast cancer cells. However, the group treated with the fCD64_mGFP exosomes loaded with the same amount of doxorubicin (shown as “Dox-exo” in FIG. 11) exhibited superior antitumor or anticancer effects compared to treatment with doxorubicin alone (FIG. 11). This result indicates that the doxorubicin-fCD64_mGFP exosomes more effectively delivered doxorubicin to the cancer cells than doxorubicin alone.

### Example 7-2: Co-Administration of Doxorubicin-fCD64_mGFP Exosomes and Anti-EGFR Antibody

In this Example, the doxorubicin-fCD64_mGFP exosomes were co-administered with an anti-EGFR antibody capable of targeting EGFR, a cancer cell-specific target, to evaluate the anti-tumor or anti-cancer effects thereof.

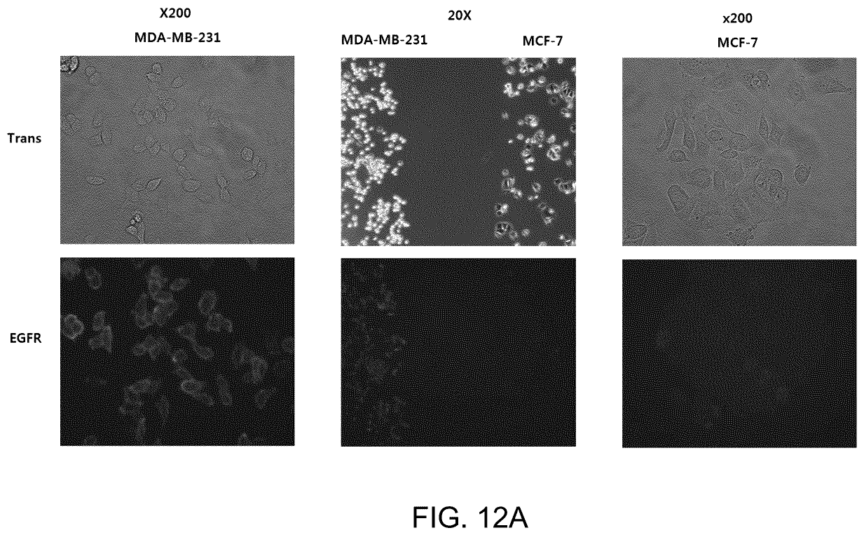

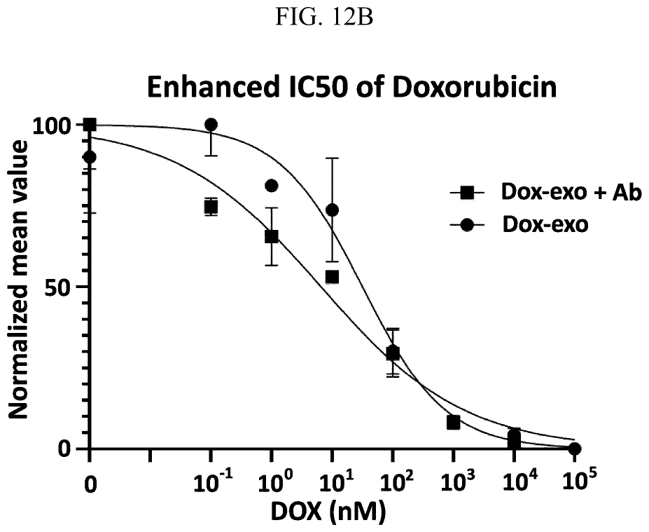

MDA-MB-231 human breast cancer cells strongly express EGFR on their surface. MDA-MB-231 cells expressing EGFR were co-cultured with MCF-7 cells not expressing EGFR, and then the cells were treated with the fCD64_mGFP exosomes tagged with a fluorescently labeled anti-EGFR antibody. As a result, it was confirmed that more exosomes were uptaken into the MDA-MB-231 cells (FIG. 12A). This result indicates that CD64 artificially displayed on the fCD64_mGFP exosome normally binds to the Fc domain of the anti-EGFR antibody.

In addition, if co-administration of the doxorubicin-fCD64_mGFP exosomes and anti-EGFR exhibits more effective antitumor or anticancer effects, this suggests that CD64 displayed on the doxorubicin-fCD64_mGFP exosomes of the present invention effectively binds to the Fc domain of anti-EGFR, and that the doxorubicin-fCD64_mGFP exosome-anti-EGFR complex targets cancer cells effectively and efficiently. In order to confirm this suggestion, an experiment was performed as follows.

10 μg of anti-EGFR was mixed with the doxorubicin-fCD64_mGFP exosomes and then incubated using a hulamixer sample mixer (purchased from Thermofisher, USA) at 4° C. for 2 hours. Thereafter, the antibody that was not specifically bound to the doxorubicin-fCD64_mGFP exosomes was removed by ultra-high-speed centrifugation, and anti-EGFR antibody-bound exosomes (hereinafter referred to as “doxorubicin-fCD64_mGFP exosomes-anti-EGFR”) were used after mixing with DPBS buffer filtered through a 0.1-μm syringe filter (product name: SLVVR33RS; purchased from Merck, Germany).

The MDA-MB-231 human breast cancer cell line (10,000 cells/well) was treated in vitro with each of doxorubicin-fCD64_mGFP exosome and doxorubicin-fCD64_mGFP exosomes-anti-EGFR. Here, the breast cancer cells were treated with each of the doxorubicin-fCD64_mGFP exosomes (shown as “Dox-exo” in FIG. 12B) and the doxorubicin-fCD64_mGFP exosome-anti-EGFR antibody (shown as “Dox-exo+Ab” in FIG. 12B) at a concentration of 2.56×10¹¹ particles/mL, and the amount of doxorubicin loaded into the doxorubicin-fCD64_mGFP exosomes was the same as the amount of doxorubicin loaded into the doxorubicin-fCD64_mGFP exosome-anti-EGFR.

As a result of the above experiment, it was confirmed that both groups synergistically inhibited the growth of the cancer cells, but the group treated with the doxorubicin-fCD64_mGFP exosome-anti-EGFR had superior anti-tumor or anti-cancer effects compared to the group treated with the doxorubicin-fCD64_mGFP exosomes (FIG. 12B). From this result, it can be seen that when cancer cells are treated with a combination of the doxorubicin-fCD64_mGFP exosomes and the anti-EGFR antibody, CD64 displayed on the doxorubicin-fCD64_mGFP exosomes effectively binds to the Fe domain of the anti-EGFR, and thus the doxorubicin-fCD64_mGFP exosome-anti-EGFR complex targets the cancer cells effectively and efficiently.

Although the present invention has been described with reference to the embodiments, the scope of the present invention is not limited to these embodiments. Any person skilled in the art will appreciate that various modifications and changes are possible without departing from the spirit and scope of the present invention and these modifications and changes also fall within the scope of the present invention.

We claim:

1. A method for producing exosomes containing an overexpressed CD64 or a portion thereof, the method comprising:
preparing a genetic construct encoding a full-length CD64 or a portion thereof, without fusion with a nucleic acid sequence encoding an exogenous transmembrane protein or a transmembrane domain thereof, wherein the portion has a transmembrane protein of CD64 or a transmembrane domain of CD64;
transducing the genetic construct into cells that do not naturally express CD64 or a portion thereof;
expressing the full-length CD64 or the portion thereof in the cells;
culturing the cells in a medium; and
isolating exosomes containing the full-length CD64 or the portion thereof.

2. The method of claim 1, wherein the exosomes are secreted or released from the cells and contained in the cell culture medium.

3. The method of claim 2, wherein the exosomes are isolated from the cell culture medium.

4. The method of claim 1, wherein the full-length CD64 or the portion thereof is located on a surface of the exosomes.

5. The method of claim 1, wherein the cells are HEK293 cells, HEK293T cells, or Expi293F cells.

6. A pharmaceutical composition comprising exosomes containing an overexpressed CD64 or a portion thereof, as an active ingredient, wherein the exosomes are prepared according to the method of claim 1.

7. The pharmaceutical composition of claim 6, comprising the exosomes and a pharmaceutically acceptable carrier.

8. The pharmaceutical composition of claim 7, wherein the composition enhances an anti-tumor effect or an anti-cancer effect.

9. The pharmaceutical composition of claim 8, further comprising a cancer cell killing agent.

10. The pharmaceutical composition of claim 9, wherein the cancer cell killing agent is located on a surface of the exosomes or inside the exosomes.

11. The pharmaceutical composition of claim 10, wherein the cancer cell killing agent is fused to the overexpressed CD64 or the portion thereof.

12. A drug delivery system comprising the pharmaceutical composition of claim 6.

13. The drug delivery system of claim 12, further comprising an antibody.

14. The drug delivery system of claim 13, wherein the antibody is co-administered with the exosomes to a subject in need thereof.

15. A cosmetic composition comprising exosomes containing an overexpressed CD64 or a portion thereof, wherein the exosomes are prepared according to the method of claim 1.

16. The cosmetic composition of claim 15, wherein the composition is a patch, a mask pack, a mask sheet, a skin softener, a nutrition, an astringent lotion, an essence, an eye essence, a cleansing foam, a cleansing water, a sunscreen, a lipstick, a bath preparation, a body essence, a body cleanser, a hairdye, a shampoo, a soap, a surfactant-containing cleanser, a cream, a lotion, an ointment, a tonic, a suspension, an emulsion, a paste, a gel, an oil, a pack, a spray, an aerosol, a mist, a foundation, a powder or an oilpaper.
